(19) United States
(12) Patent Application Publication
(10) Pub. No.: US 2022/0371991 A1
(43) Pub. Date: Nov. 24, 2022

(54) SYNTHESIS OF A POLYURETHANE FOAM INCORPORATING INDUSTRIAL BYPRODUCTS OR WASTE

(71) Applicant: Sohi PATEL, Houston, TX (US)

(72) Inventor: Sohi PATEL, Houston, TX (US)

(21) Appl. No.: 17/307,856

(22) Filed: May 4, 2021

(51) Int. Cl. C07C 263/02; C08J 9/02; C08G 18/73; C08G 18/32; C07H 3/04

(52) U.S. Cl. CPC C07C 263/02; C08J 9/02; C08G 18/73; C08G 18/3206; C07H 3/04; C08J 2375/04

(57) ABSTRACT

A process includes calcining a high potassium carbonaceous waste product to form potassium oxide and carbon dioxide and reacting the potassium oxide and carbon dioxide to yield bio-based potassium carbonate. The process also includes catalyzing a reaction of a lignocellulosic biomass with abundant hydroxyl groups into a biopolyol using the bio-based potassium carbonate and brominating using a brominating agent a triglyceride to form a bio-isocyanate. In addition, the process includes reacting the biopolyol and the bio-isocyanate to form a polyurethane foam.

Embodiments of the present disclosure generally relate to the synthesis of a polyurethane foam and articles made therefrom.

### BACKGROUND

Conventional polyurethane foam is traditionally formed of polyol and isocyanate units joined by carbamate links and blown by a natural carboxyl agent. One traditional method of forming isocynates is through phosgenation. The phosgenation process is hazardous due the use of methyl isocyanate gas. Conventional polyurethane foam emits VOC long after application.

Polyols, a component of polyurethane foam production, are derived from petrochemicals and are thus, nonrenewable. Previous efforts to produce renewable polyols from biomaterials have been largely unsuccessful. Polyols may be derived from natural oils, such as canola oil, and then used in polyurethane foam production; the pre-existing carbon-carbon double bonds in natural oils are readily introduced to hydroxyl groups (—OH) through oxidation and, thereafter, converted to diols or polyols. However, most natural oils used are drying or semi-drying oils, which impede oxidative cross-linking and bond-breaking. Therefore, polyurethane foams formed from such polyols are difficult to be completely degraded or completely cured. Furthermore, methods of forming polyurethane foams are traditionally multi-step, multi-pot oxidation reactions and are inefficient.

### BRIEF DESCRIPTION OF THE DRAWINGS

The present disclosure is best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion.

FIG. 1 is a graphical depiction of TVOC emission levels of synthesized polyurethane foam samples over time.

FIG. 2 is graphical depiction of biodegradation of synthesized polyurethane foam samples over time.

### SUMMARY

A process for forming a biopolyol is disclosed. The process includes calcining a high potassium carbonaceous waste product to form potassium oxide and carbon dioxide and reacting the potassium oxide and carbon dioxide to yield bio-based potassium carbonate. The process also includes catalyzing a reaction of a lignocellulosic biomass with abundant hydroxyl groups into a biopolyol using the bio-based potassium carbonate.

A process for forming a bio-isocyanate is disclosed. The process includes brominating using a brominating agent a triglyceride to form a bio-isocyanate.

In addition, a process for forming a polyurethane foam is disclosed. The process includes calcining a high potassium carbonaceous waste product to form potassium oxide and carbon dioxide and reacting the potassium oxide and carbon dioxide to yield bio-based potassium carbonate. The process also includes catalyzing a reaction of a lignocellulosic biomass with abundant hydroxyl groups into a biopolyol using the bio-based potassium carbonate and brominating using a brominating agent a triglyceride to form a bio-isocyanate. In addition, the process includes reacting the biopolyol and the bio-isocyanate to form a polyurethane foam.

### DETAILED DESCRIPTION

A detailed description will now be provided. The following disclosure includes specific embodiments, versions and examples, but the disclosure is not limited to these embodiments, versions or examples, which are included to enable a person having ordinary skill in the art to make and use the disclosure when the information in this application is combined with available information and technology.

Various terms as used herein are shown below. To the extent a term used in a claim is not defined below, it should be given the broadest definition persons in the pertinent art have given that term as reflected in printed publications and issued patents. Further, unless otherwise specified, all compounds described herein may be substituted or unsubstituted and the listing of compounds includes derivatives thereof.

Further, various ranges and/or numerical limitations may be expressly stated below. It should be recognized that unless stated otherwise, it is intended that endpoints are to be interchangeable. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

Certain embodiments of the present disclosure relate to a biopolyol. Other embodiments of the present disclosure relate to a bio-isocyanate chemical. Certain other embodiments of the present disclosure relate to a process of forming a bio-based polyurethane foam system.

### Catalyst Manufacture

In certain embodiments, a biopolyol catalyst may be formed by calcining a high potassium carbonaceous waste product, for example, but not limited to, orange peel, watermelon peel, pineapple peel, banana peel, and cacao peel, to form potassium oxide and carbon dioxide. The potassium oxide and carbon dioxide may be reacted to yield bio-based potassium carbonate.

### Formation of the Biopolyol

In some embodiments, the bio-based potassium carbonate may be used as a catalyst to convert a lignocellulosic biomass with abundant hydroxyl groups into a biopolyol through delignification and repolymerization as shown in equation 1.

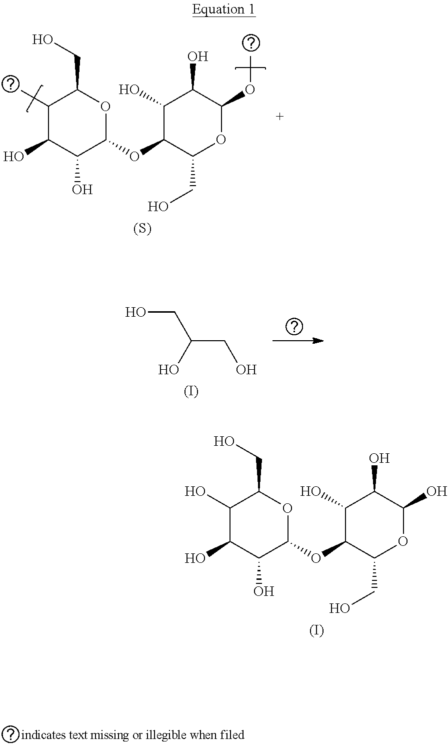

Examples of bio-based components with abundant hydroxyl groups include soybean straw, corn stover, rice stalk, wheat straw or wheat bran, wood chips, sawdust or any wood waste or grain-like agricultural material. In certain embodiments, the bio-based component with abundant hydroxyl groups may be milled and liquefied prior to reaction.

### Bio-Isocyanate Synthesis

In some embodiments of the present disclosure, a bio-isocyanate may be formed using a triglyceride high in linoleic acid content, including, but not limited to, unsaturated plant oils such as soybean oil, linseed oil, and corn oil. During bio-isocyanate synthesis, the triglyceride is brominated at the allylic positions to form an allylic bromide, such as, for example, allylic brominated soybean oil. Bromination reagents may include, for example, N-bromosuccinimide. In certain embodiments, bromination may be performed in a solvent, for example, acetonitrile, carbon tetrachloride, or benzotrifluoride. In some embodiments, carbon tetrachloride and benzotrifluoride may not be used because of toxicity or lack of availability. The allylic bromide may be replaced with a cyanate group through a substitution reaction to yield a bio-isocyanate unit, as shown in Equation 2. During the substitution reaction, allylic bromide may be dissolved in, for example, a polar aprotic solvent such as tetrahydrofuran, dichloromethane, acetonitrile, or ethyl acetate. In certain embodiments, the solvent for the substitution reaction and the bromination procedure is a different solvent. The cyanate group may be supplied by, for example, silver cyanate.

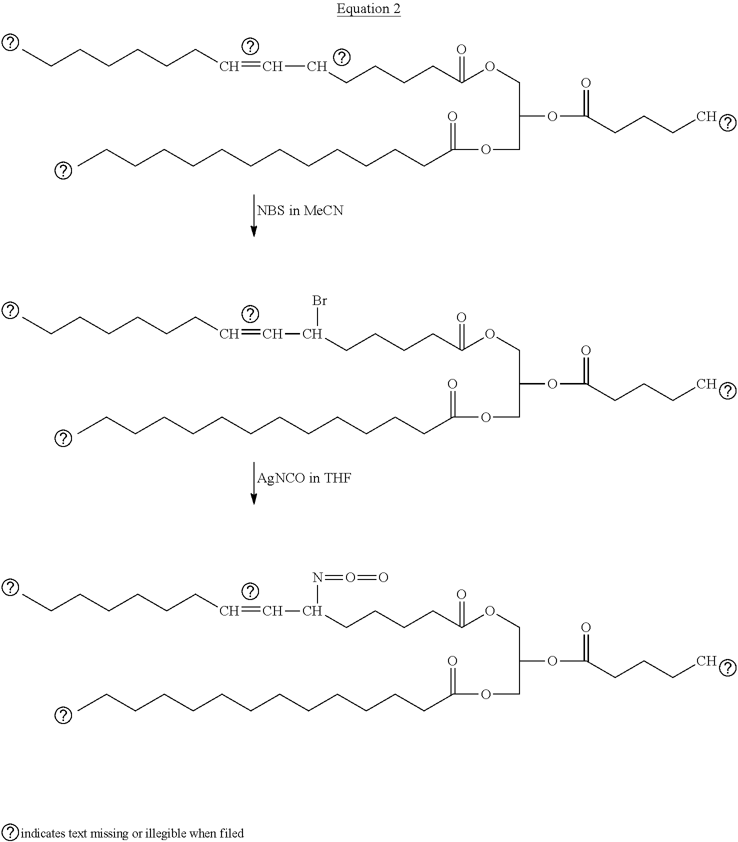

### Polyurethane Synthesis

As shown in Equation 3, the biopolyol and the bio-isocyanate may be reacted to form a polyurethane foam.

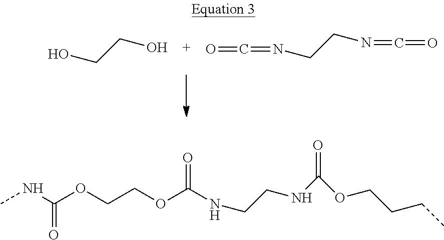

### EXAMPLES

The disclosure having been generally described, the following examples show particular embodiments of the disclosure. It is understood that the example is given by way of illustration and is not intended to limit the specification or the claims. All compositions percentages given in the examples are by weight.

### Example 1—Catalyst Manufacture

20.0 grams of orange peels were dried and calcined at 600° C. for 1 hour. The obtained white ash was washed with 283 milliliters of deionized water and filtered twice to yield 8.03 grams of bio-based potassium carbonate.

### Example 2—Biopolyol Formation

20.0 grams of soybean straw were milled and liquefied for 1.5 hours at solvent reflux; 200 milliliters of crude glycerol served as the liquefaction solvent and 6.60 grams of bio-based potassium carbonate served as the liquefaction catalyst to yield 145 milliliters of biopolyol. 37.5 milliliters of trace methanol were recovered from the crude glycerol solvent during liquefaction.

### Example 3—Bio-Isocyanate Synthesis

36.0 grams of soybean oil were stirred with 45.0 grams of N-bromosuccinimide in 270 milliliters of acetonitrile at solvent reflux. After resting overnight, the solvent was evaporated under reduced pressure for 2 hours to yield 62.0 grams of allylic brominated soybean oil (ABSO). 270 milliliters of the acetonitrile solvent were recovered during rotary evaporation.

8.00 grams of ABSO were then dissolved in 20.0 milliliters of tetrahydrofuran (THF). 66.0 milliliters of silver cyanate slurry, including 6.00 grams of silver cyanate dissolved in 60.0 milliliters of THF, was added to the ABSO solution in three parts, with 30 minute intervals between each addition. The solution was stirred for 3 more hours and rested for 10 hours before yielding 210 milliliters of bio-isocyanate. 10.2 milliliters of silver bromide slurry were recovered as a pale cream precipitate.

### Example 4—Polyurethane Synthesis

12.0 milliliters of polyether polyol system were reacted with 18.0 milliliters of multi-isocyanate system to yield a polyurethane foam system, labeled “Sample A”. 12.0 milliliters of polyether polyol system reacted with 18.0 milliliters of bio-isocyanate to yield a partially bio-based polyurethane foam system, labeled “Sample B”. 12.0 milliliters of biopolyol reacted with 18.0 milliliters of multi-isocyanate system to yield another partially bio-based polyurethane foam system, labeled “Sample C”. 12.0 milliliters of biopolyol reacted with 18.0 milliliters of bio-isocyanate to yield a fully bio-based polyurethane foam system, labeled “Sample D”.

### Example 5—Comparison of Samples

Conventional foam characterization tests, such as compression set method (1), thermal and humid aging (2), and water absorption method (3) were performed on each polyurethane foam sample.

Compression set was calculated by applying 2.27 kilograms of weight to 4×4 centimeter slabs of each foam sample for 1 minute each. After the weight was removed, each sample was allowed 1 minute to rest before compression and deflection measurement. Humid and thermal aging were determined by heating either wet or dry 5-gram slabs of each foam sample at 100° C. for 2 hours, respectively. Water absorption was measured gravimetrically during wet slab preparation. 50-gram slabs of each foam sample were placed in separate airtight containers, and total volatile organic chemical emissions were measured with a TVOC sensor over the course of 5 days. 4-gram slabs of each foam sample were incubated in separate soil immersion chambers, and biodegradability was measured gravimetrically over the course of 10 days. Cell size was measured through stereo microscopy.

$$Compression Set = ( \frac{( initial thickness - final thickness )}{( initial thickness - space bar thickness )} ) * 100 \qquad ( 1 )$$

$$Rate of Aging = \frac{( final weight - initial weight )}{total hours aged} \qquad ( 2 )$$

$$Water Absorption = ( \frac{( final weight - initial weight )}{initial weight} ) * 100 \qquad ( 3 )$$

Other standards of polyurethane foam, such as foaming time, density, and cell size, were measured through common gravimetric analysis methods and simple visual or microscopic observation. Material characteristics for each polyurethane foam sample are reported below (Table 1).

Material Characteristics of Synthesized Polyurethane Foam

TABLE 1

| | Sample A | | Sample B | | Sample C | | Sample D | |
|---|---|---|---|---|---|---|---|---|
| Foaming Time | 100 | sec | 109 | sec | 176 | sec | 102 | sec |
| Density | 0.55 | g/cm³ | 0.12 | g/cm³ | 0.10 | g/cm³ | 0.52 | g/cm³ |
| Compression Set | 0% | | 0% | | 68.75% | | 0% | |
| Rate of Thermal Aging | 0 | g/hr | 0 | g/hr | 0.5 | g/hr | 0 | g/hr |
| Rate of Humid Aging | 1 | g/hr | 2 | g/hr | 7.5 | g/hr | 0 | g/hr |
| Water Absorption | 20% | | 60% | | 340% | | 0% | |
| Cell Size | 312 | μm | 253 | μm | 578 | μm | 323 | μm |

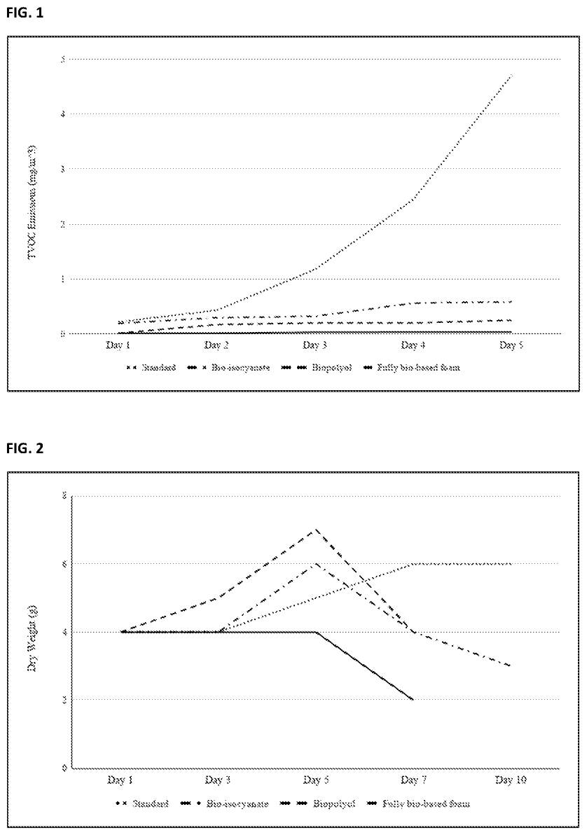

Total volatile organic chemical (TVOC) emissions were recorded for each polyurethane foam sample as shown in FIG. 1. Short-term foam biodegradability was also measured through a conventional soil immersion technique as shown in FIG. 2. Data collection prematurely terminated for Samples C & D as the immersed foam slabs became indistinguishable from soil media during biodegradation.

In addition to material testing for each polyurethane foam sample, certain measures of green chemistry, such as atom economy (4) and percentage yield (5) were calculated for the synthesized biopolyol, bio-isocyanate, and other intermediary biochemicals.

$$Atom Economy = ( \frac{FW of atoms utilized}{FW of all reactants} ) * 100 \qquad ( 4 )$$

$$“ FW ” indicates formula weight .$$

$$Percentage Yield = ( \frac{actual yield}{theoretical yield} ) * 100 \qquad ( 5 )$$

Standard scalability and sustainability characteristics of synthesized biochemicals derived from these metrics are indicated in Table 2.

TABLE 2
Scalability and Sustainability Characteristics
of Synthesized Biochemicals

| | | | | Bio- |
|---|---|---|---|---|
| | Bio K₂CO₃ | Biopolyol | ABSO | Isocyanate |
| Atom Economy | 97% | 100% | 65% | 75% |
| Percentage Yield | 78.13% | 90.29% | 96.84% | 240.13% |
| Reaction | GOOD | EXCEL- | EXCEL- | SUPERIOR |
| Efficiency | | LENT | LENT | |

Three distinct chi-square tests of independence (6) were performed to determine the difference in material performance between standard polyurethane foam, yielding expected values, and partially or fully bio-based polyurethane foam, yielding observed values as shown in Table 3.

$${X}^{2} = \sum \frac{{( o - e )}^{2}}{e} \qquad ( 6 )$$

Note. In this context, “o” indicates the observed value and “e” indicates the expected value.

TABLE 3
Material Performance Chi-Square Crosstabulation

| Foam | | | Chi-square |
|---|---|---|---|
| composition | | Material Performance | stat, |
| (ternary) | | Values | p-value |
| SAMPLE B: | Observed | 109, 0.12, 0, 0, 2, 60, 253 | χ = 9.661 |
| Biopolyol, | Expected (A) | 100, 0.55, 0, 0, 1, 20, 312 | p = .139673 |
| Standard | Residual | 9, −0.43, 0, 0, 1, 40, −59 | |
| isocyanate | | | |
| SAMPLE C: | Observed | 176, 0.10, 68.75, 0.5, 7.5, | χ = 73.805 |
| Standard | Expected (A) | 340, 578 | p < .00001 |
| polyol, | Residual | 100, 0.55, 0, 0, 1, 20, 312 | |
| Bio-isocyanate | | 76, −0.45, 68.75, 0.5, 6.5, | |
| | | 320, 266 | |
| SAMPLE D: | Observed | 102, 0.52, 0, 0, 0, 0, 323 | χ = 4.629 |
| Biopolyol, | Expected (A) | 100, 0.55, 0, 0, 1, 20, 312 | p = .592197 |
| Bio-isocyanate | Residual | 2, −0.03, 0, 0, −1, −20, 11 | |

An additional three distinct chi-square tests of independence were performed to determine the difference in environmental safety between standard polyurethane foam and partially or fully bio-based polyurethane foam (Table 4).

TABLE 4
Environmental Safety Chi-Square Crosstabulation

| Foam | | | Chi-square |
|---|---|---|---|
| composition | | Environmental | stat, |
| (ternary) | | Safety Values | p-value |
| SAMPLE B: | Observed | 0.587, 83.3 | χ = 27.743 |
| Biopolyol, | Expected (A) | 4.716, 7.5 | p < .00001 |
| Standard isocyanate | Residual | −4.129, 75.8 | |
| SAMPLE C: | Observed | 0.251, 69.6 | χ = 22.769 |
| Standard polyol, | Expected (A) | 4.716, 7.5 | p < .00001 |
| Bio-isocyanate | Residual | −4.465, 62.1 | |
| SAMPLE D: | Observed | 0.035, 94.5 | χ = 31.841 |
| Biopolyol, | Expected (A) | 4.716, 7.5 | p < .00001 |
| Bio-isocyanate | Residual | −4.681, 87 | |

Depending on the context, all references herein to the “disclosure” may in some cases refer to certain specific embodiments only. In other cases it may refer to subject matter recited in one or more, but not necessarily all, of the claims. While the foregoing is directed to embodiments, versions and examples of the present disclosure, which are included to enable a person of ordinary skill in the art to make and use the disclosures when the information in this patent is combined with available information and technology, the disclosures are not limited to only these particular embodiments, versions and examples. Other and further embodiments, versions and examples of the disclosure may be devised without departing from the basic scope thereof and the scope thereof is determined by the claims that follow.

What is claimed is:

1. A process for forming a biopolyol comparing:
calcining a high potassium carbonaceous waste product to form potassium oxide and carbon dioxide;
reacting the potassium oxide and carbon dioxide to yield bio-based potassium carbonate;
catalyzing a reaction of a lignocellulosic biomass with abundant hydroxyl groups into a biopolyol using the bio-based potassium carbonate.

2. The process of claim 1, wherein the high potassium carbonaceous waste product is orange peel, watermelon peel, pineapple peel, banana peel, and cacao peel.

3. The process of claim 1, wherein the lignocellulosic biomass with abundant hydroxyl groups is soybean straw, corn stover, rice stalk, wheat straw, wheat bran, wood chips, sawdust.

4. A process for forming a bio-isocyanate comprising:
brominating using a brominating agent a triglyceride to form a bio-isocyanate.

5. The process of claim 4, wherein the triglyceride is soybean oil, linseed oil, and corn oil.

6. The process of claim 4, wherein the brominating agent is N-bromosuccinimide.

7. A process for forming a polyurethane foam comprising:
calcining a high potassium carbonaceous waste product to form potassium oxide and carbon dioxide;
reacting the potassium oxide and carbon dioxide to yield bio-based potassium carbonate;
catalyzing a reaction of a lignocellulosic biomass with abundant hydroxyl groups into a biopolyol using the bio-based potassium carbonate
brominating using a brominating agent a triglyceride to form a bio-isocyanate;
reacting the biopolyol and the bio-isocyanate to form a polyurethane foam.
